(19) United States
(12) Patent Application Publication
(10) Pub. No.: US 2014/0081206 A1
(43) Pub. Date: Mar. 20, 2014

(54) Medical Assembly Comprising Monitoring Device

(71) Applicants: Marcel Mueller, Therwil (CH); Edgar Jeanbourquin, Ruefenacht (CH); Florian Zoergiebel, Bern (CH); Franco Moia, Frenkendorf (CH)

(72) Inventors: Marcel Mueller, Therwil (CH); Edgar Jeanbourquin, Ruefenacht (CH); Florian Zoergiebel, Bern (CH); Franco Moia, Frenkendorf (CH)

(73) Assignee: ROCHE DIAGNOSTICS INTERNATIONAL AG, Rotkreuz (CH)

(21) Appl. No.: 13/906,492

(22) Filed: May 31, 2013

Related U.S. Application Data
(63) Continuation of application No. PCT/EP2011/070974, filed on Nov. 24, 2011.

(30) Foreign Application Priority Data
Dec. 6, 2010 (EP) 10193837.1

(51) Int. Cl. A61M 5/50; A61M 5/168

(52) U.S. Cl. CPC A61M 5/5086; A61M 5/168
USPC 604/111; 29/428

(57) ABSTRACT

A method of preparing at least part of a medical device for application and a medical assembly for preparing or storing the at least one disposable unit of the medical device is presented. The medical assembly comprises a chemical or physical monitoring device for counting and/or displaying a time period of application and/or time remaining before required disposal or replacement of the disposable unit. The monitoring device is automatically activated by moving the at least one disposable unit relative to a second unit of the medical assembly for preparing or positioning the disposable unit for use with the medical device; assembling the at least one disposable unit and the medical device; and/or withdrawing the disposable unit of a storage compartment of the second unit.

### CROSS-REFERENCE TO RELATED APPLICATIONS

This application is a continuation of PCT/EP2011/070974, filed Nov. 24, 2011, which is based on and claims priority to EP 10193837.1, filed Dec. 6, 2010, which is hereby incorporated by reference.

### BACKGROUND

The present disclosure generally relates to a medical assembly comprising at least one disposable unit and, in particular, to a medical assembly comprising at least one disposable unit such as an infusion set assembly comprising a disposable infusion head and a monitoring device for counting and/or displaying a time period of application and/or time remaining before disposal or replacement of the disposable or replaceable unit as well as a method of preparing a medical assembly for use.

It is a well-known problem in continuous, or repetitive use, medical devices that, in practice, it is difficult to guarantee that disposable materials of the medical device are changed within a time period necessary for reliable operation of the medical device. This is, for example, a relevant aspect of medical devices 1) for administration of medical, or pharmaceutical preparations, using disposable injection or infusion units, 2) for measuring health data of a patient using disposable measuring units, 3) for treatment of different patients by the same medical device using disposable or replaceable tool units or 4) even simply for storing disposable or replaceable units of medical devices or parts thereof. An application period and/or expiry date of disposable units is often only provided on the outside of a packaging, whereas the unit itself is not provided with these dates. A specific field of use of medical devices comprising disposable or replaceable units are infusion sets for an insulin pump therapy, in which for example infusion sets in most cases are replaced by diabetics themselves or their relatives. Even if these persons are not especially handicapped and despite the fact that they are specifically trained, they often forget to replace disposable units of their medical device, resulting in reduced insulin absorption of the patient's tissue and complications such as for example, inflammation or repulsion reactions.

Solutions known in the art include noting relevant data an a separate protocol sheet, providing passive visible indicators also in combination with calendars and the like, for example, a device similar to a parking disk, or disabling a medical device electronically if for example one unit of the medical device comprises an integral data memory. Also a variety of electronic auxiliaries for medical devices are known, which either are provided as additional programming or control units, like computers, or may be integrated in a medical device using sensors, processors, LEDs and the like.

On the other side, manifold medical devices are known from the art, which include indication or alarm devices for indicating a failure of the medical device. In particular in the field of medical pump systems alarm devices for indicating leakage, blockage or air enclosure problems are known. The principles of indicating or alerting a user of a medical assembly or a medical device comprising disposable or replaceable units known from the art are generally realized either by complex electronic means or by very simple labeling, which is missing relation to the actual circumstances of application of the medical device. Often the indication devices are not directly linked with the disposable or replaceable unit or part of the medical assembly or the indication devices refer to the durability of materials or medications used but not to an optimum application period of a disposable unit, which interacts with its environment during application.

Therefore, there is a need for a method of preparing a medical assembly, a medical device or at least part thereof, which facilitates handling and preparation, enables simple application and use of the medical device, makes maintenance easier and increases convenience of a person applying the medical assembly or medical device with reliable indication of an application period or ending of an application period for such a unit, which indicates the status of such unit irrespective of its age, which offers easy inspection of a time period an such unit, which does not require electric or electronic auxiliaries and which offers easy handling.

### SUMMARY

According to the present disclosure, a disposable medical device for use in combination with a further medical device and a method of use are presented. The medical device can comprising a monitoring device for counting and/or displaying a time period of application and/or time remaining before required disposal or replacement of the disposable or replaceable unit, the monitoring device switches, upon activation, from an inactive state to an active state where its counts and/or displays the time period of application and/or time remaining before required disposal. The monitoring device can be automatically activated along with carrying out a preparatory or handling step for preparing the disposable medical device for use.

Accordingly, it is a feature of the embodiments of the present disclosure to provide a method of preparing a medical assembly, a medical device or at least parts thereof, which facilitates handling and preparation, enables simple application and use of the medical device, makes maintenance easier and increases convenience of a person applying the medical assembly or medical device with reliable indication of an application period or ending of an application period for such a unit, which indicates the status of such unit irrespective of its age, which offers easy inspection of a time period an such unit, which does not require electric or electronic auxiliary means and which offers easy handling. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1a illustrates schematic view of a medical assembly in a first state from a first side according to an embodiment of the present disclosure.

FIG. 1b illustrates schematic view of the medical assembly according to FIG. 1a in the first state from a second side according to an embodiment of the present disclosure.

FIG. 1c illustrates schematic view of the medical assembly according to FIG. 1a in a second state according to an embodiment of the present disclosure.

FIG. 2a illustrates schematic view of a medical assembly in a first state according to another embodiment of the present disclosure.

FIG. 2b illustrates schematic view of a medical assembly according to FIG. 2b in a second state according to an embodiment of the present disclosure.

FIG. 3a illustrates three-dimensional sectional view of a medical assembly according to yet another embodiment of the present disclosure.

FIG. 3b illustrates three-dimensional sectional view of a medical assembly connected to a medical device according to another embodiment of the present disclosure.

FIG. 4a illustrates schematic view of a fourth embodiment of a medical assembly in a first state according to an embodiment of the present disclosure.

FIG. 4b illustrates schematic view of a fourth embodiment of a medical assembly in a first state according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A disposable medical device or unit for use in combination with another medical device or unit can comprise a monitoring device for counting and/or displaying a time period of application and/or time remaining time before required disposal or replacement of the disposable unit. The monitoring device can switch, upon activation, from an inactive state to an active state, where it can count and/or display the time period of application and/or remaining time before required disposal. The disposable medical device or unit can be designed such that the monitoring device can be automatically activated along with carrying out a preparatory or handling step that can be required for preparing the disposable medical device for use.

A method of preparing at least part of a medical device for application can refer to medical devices comprising at least one disposable unit, like a disposable medical device, which has to be prepared for use and/or application in combination with the medical device or for integration into the device. The at least one disposable unit may be part of a medical assembly for preparing or storing the at least one disposable unit. Furthermore, the medical device, in particular the at least one disposable device or unit, can comprise a chemical or physical monitoring device for counting and/or displaying a time period of application and/or time remaining before required disposal or replacement of the disposable unit. This can mean the monitoring device can indicate a time period, which can start with the beginning of the preparation of the disposable unit or at the beginning of the application of the disposable unit by the medical device.

In the field of medical devices, a disposable unit can be understood as a unit or a part of the medical device, which can be discarded or replaced after a certain time of use because of medical, safety, hygienic, or other reasons. A disposal of such units or parts can be highly recommended to ensure proper working of the medical device and avoid negative effects on the patients. Other parts or units of the medical device may be reusable, which means they may be used repeatedly with different disposable units. Disposable units can be for example infusion sets, injection needles, lancets, cartridges and so on. Depending an the function of the disposable unit, it can be replaced or disposed after a few hours, a few days or even a few weeks.

According to one embodiment, the monitoring device can be automatically activated by moving the at least one disposable device or unit relative to a second unit of the medical assembly for preparing or positioning the disposable device or unit for use with the medical device; for assembling the at least one disposable device or unit and the medical device; and/or for withdrawing the disposable device or unit of a storage compartment of the second unit. Therefore the monitoring device cannot be activated by a particular individual activating process with the only intent of starting the monitoring device. Rather the monitoring device can be activated by a handling or preparation step, which can be mandatory within the regular use and preparation of the medical device for use and which can be compulsory accomplished for the designated application of the medical device. Such designated application can be for example an infusion of fluids, measuring patient data, or the like. In contrary to a mandatory and compulsory step, the mere step of activating the monitoring device may not be required for the intended use of the medical device, which can mean the device can work properly without activating the monitoring device.

Since the activation of the monitoring device can be linked to a compulsory preparation step of the medical device, the activation of the monitoring device or associated registration of relevant data for carrying out correct disposal or replacement of the disposable unit may not be forgotten or unintentionally ignored by a patient. Also the method does not only rely an complex electric or electronic systems requiring an energy source. The method can be simple, reliable and convenient for a patient.

A medical assembly can comprise at least two units moveable relative to each other. At least one of the units can be a disposable device or unit designed for application in combination with a medical device or for integration into a medical device. Furthermore, the medical assembly can comprise a chemical or physical monitoring device for counting and/or displaying a time period of application and/or time remaining before required disposal or replacement of the disposable or replaceable unit. This can mean the monitoring device can indicate a remaining time period before the disposable device or unit has the be substituted or a time period, which elapsed since the beginning of preparation of the medical device and the disposable device or unit respectively. In an inactive state of the monitoring device or the disposable device or unit respectively, the at least two units can take a first position relative to each other and the monitoring device can be inactive. In an active state, the at least two units can take a second position after the units have been moved relative to each other and the monitoring device is active. The monitoring device can be automatically activated to count and/or display the time period by movement of the at least two units relative to each other from the first to the second position. The automatic activation can be a movement of the at least two units, which can be mandatory for correct preparation or application of the medical device. The activation can be simultaneously, i.e. automatically, with the obligatory movement of the units of the medical assembly to prepare the disposable device or unit for the medical device.

In one embodiment of a medical assembly, the monitoring device can be on the disposable or replaceable unit, for example an infusion head unit for a medical infusion device. The monitoring device can comprise a pressure sensitive activation mechanism, so that the monitoring device may be activated be generating pressure on at least parts of the monitoring device, for example, an interaction area on one of the at least two units. This can be accomplished for example by connecting a coupler port to an infusion device. The pressure may be generated by frontal abutment or by sliding one upon the other of the at least two units, wherein the monitoring device can be at least partly squeezed between the two units.

For example, the monitoring device can comprise at least one activating chamber including a first reactant and at least one indication chamber including a second reactant. The activating chamber and the indication chamber can be separated in the inactive state of the monitoring device and can be connected in the active state. In general the activating chamber of the monitoring device can be located in the interaction area, such that the first or second unit can act on the interaction area and can activate the monitoring device, when moved from first to second position. The interaction area may be parallel or perpendicular to the moving direction of the at least two units relative to each other. A connecting conduit can be present between the activating chamber and the indication chamber, which can be blocked in inactive state of the monitoring device and which can be unblocked in the active state. The connecting conduit may be a simple passage, by a membrane, by a predetermined breaking point, or the like. The blocking can be overcome by generating a pressure in the activation chamber, for example, by pressing the second unit on the interaction area with the activation chamber. The pressure can force the reactant through the connecting conduit against a blocking force, for example, a breaking point, membrane resistance, capillary force or the like, or can interrupt a separator arranged between the activating chamber and the indication chamber. The separator can be within the connecting conduit.

Alternatively, the blocking of the connecting conduit may be a separator arranged on the second unit of the medical assembly, which can interact with the monitoring device or the connecting conduit respectively on the disposable unit and can block the conduit from outside the monitoring device. Such a separator may be removed from the connecting conduit by the relative movement of the disposable unit relative to the second unit of the medical assembly.

After the connecting conduit has been unblocked, the first reactant of the activating chamber can flow towards the indication chamber and first and second reactants can react with each other such that a visible indication can result in the indication chamber. The visible indication can move along an axis of the indication chamber in a predetermined period of time. The movement can correspond to continued reaction of the reactants. Next to the indication chamber, a time scale may be provided, which can assign the position of the visible indication within the indication chamber to a period of time since the separation of the chambers has been abolished. Thus the time scale can indicate the period of time since the at least two units of the medical assembly have been moved relative to each other to prepare the medical device for application. Also the time scale may indicate the remaining time until the disposable unit has to be removed from the medical device, for example, by a remaining distance to be overcome by the visual indication within the indication chamber. The visual indication, for example, can be the front line of a color area moving forward within the indication chamber during continuous reaction of first and second reactant.

The time scale may indicate the time that lapsed since activation of the monitoring device and/or the remaining time until the disposable device may need to be discarded and replaced by a fresh one. The monitoring device and the time scale may be such that that the monitoring can end with the recommended maximum usage time. That is, if the recommended maximum usage time is, for example 3 days, the monitoring device may be designed to operate for three days. Alternatively, the monitoring device may be designed to operate for a longer time period of, for example 4 to 5 days with a recommended maximum usage time of three days. Visual indication, such as symbols or traffic light colors, may be provided as part of the time scale to indicate if the recommended maximum usage time is not yet due, is due, or is overdue.

The monitoring device can be sealed onto a surface of the disposable unit and can be covered by a tight sealing layer, which may not be interrupted during normal use of the medical device. For example, the activating chamber and the indication chamber, and if present also the connecting conduit, can be sealed onto the surface of the disposable or replaceable unit. That can mean any reactants of the monitoring device can be prevented from interacting with the medical device, the medication within the device or even the tissue of the user.

In one embodiment, the second unit may be a storage unit for storing the first unit, such as, for example, the disposable or replaceable unit. The storing unit can comprise a separator for separating the activating chamber and the indication chamber. In one embodiment, the separator can be on a base of the storing unit. The separator may be tightly attached to or integrated in the base. The monitoring device of the disposable unit can rest on the separator in the first state of the monitoring device. As soon as the disposable unit is taken off the storing unit, the monitoring device can be released from the separator and the monitoring device can start indicating the time period. In another embodiment, the separator can be on a closing foil of the storing unit and can engage with the monitoring device in a closed state of the storing unit. The closing foil may be sealed on an opening of the storing unit. Also the separator may be sealed to the closing foil. When the closing foil is stripped off the storing unit, the separator can remain on the closing foil. The disposable unit with the monitoring device can stay in the storing unit or may be lifted up together with the separator and can be detached of the closing foil. Thus the separator can be released from the monitoring device and the indication of the time period may start.

The method can be applied to a medical device, which can be an infusion device comprising a disposable unit designed as an infusion head to be placed on a tissue of a patient for administration of medical or pharmaceutical liquid into the tissue. The infusion head may comprise a connected or connectable cannula for insertion into a body tissue, which can be moveably arranged on or at the infusion head. Furthermore, the infusion head may comprise a coupling port for fluidic connection of the cannula to the infusion device. The cannula and the coupling port can be on a base element, for example, in the form of pad, which can be attached to the tissue. The monitoring device can be on the infusion head. The infusion head can be part of a medical assembly comprising at least one further unit, which can be moveable relative to the infusion head. The further unit may be designed as a preparation or storing unit interacting with the infusion head. The monitoring device can be activated by connecting the infusion head to a coupler of the infusion device; by preparing or positioning the cannula in relation to the infusion head or the infusion device; or by opening a storage unit, which can be housing the infusion head, or withdrawing the infusion head of the storage unit. These steps can be executed during the general preparation of the infusion device for application. Therefore no special attention needs to be paid to the activation of the monitoring device or to registration of data concerning the time period of application of the infusion head and the infusion device.

In one embodiment, the second unit of a medical assembly comprising a disposable or replaceable infusion head can be a coupler, which can be releasably connectable to the coupling port of an infusion head. The coupler and the coupling port can provide a fluidic connection between the infusion head and an infusion device. The coupling port and the coupler can have a connection and a counter connection designed for fluid tight engagement with each other. The engagement may be a snap connection, screw connection, a combination thereof or the like, The interaction area of the monitoring device may be on or adjacent to the coupling port or the coupler, for example, the connection or counter connection. Of course such a connection and counter connection may be realized for other medical assemblies than an infusion head or an infusion device.

In another embodiment, the medical assembly may comprise a second unit realized by an operation housing, which at least can partly cover the disposable or replaceable unit, for example, an infusion head, and can interact with a preparation mechanism of the disposable or replaceable unit, for example, a cannula mechanism, of the infusion head. Such a cannula mechanism can move the cannula relative to the infusion head from a storing position to an insertion position. In the storing position, the cannula can be protected by the operating housing or other casing of the infusion head and, in an insertion position, the cannula can be exposed and ready for insertion. Therefore, the cannula can be moved relative to the base pad of the infusion head for example by a swivel or sliding mechanism as known from the art. Such cannula mechanism can be operated by the operation housing, for example, by pushing two housing portions into each other. One of the housing portions simultaneously can press on the interaction area of the monitoring device and can start counting and indicating the time period elapsed since the cannula has been exposed. Of course the operation housing can interact with other moveable parts of a disposable unit designed for preparation of the disposable unit for application in combination with a medical device.

Another example of the method can be a medical assembly comprising a blood glucose measuring device and test strip units. The measuring strips can be disposable material and can be prepared for use with the measuring device. Also the medical assembly may comprise a case box for storing measuring strips for a blood glucose measuring device. The monitoring device can be on the case box such that it can be activated by opening and withdrawing a measuring strip.

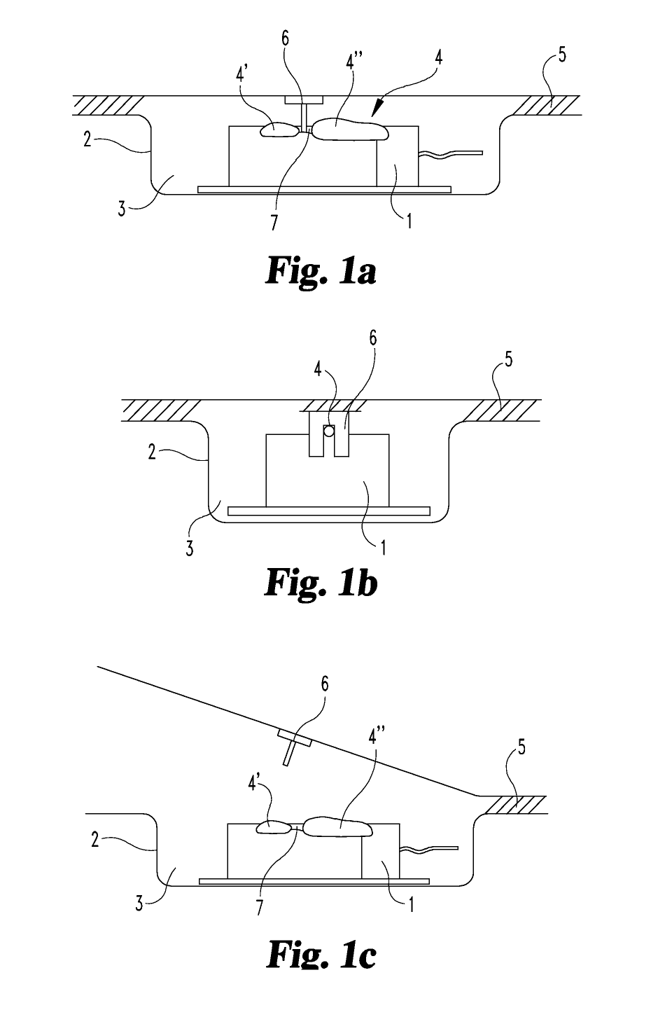

Referring initially to FIGS. 1a-c, a first embodiment of a medical assembly is shown comprising a disposable unit in form of an infusion head 1. The infusion head 1 can be stored in a second unit in form of a storing unit 2 comprising a storage compartment 3. The infusion head 1 can rest on a base of the storage compartment 3. A monitoring device 4 can be attached to the infusion head 1 on an upper side facing a closing foil 5 sealed to the storing unit 2 and closing the same. The monitoring device 4 can comprise an activating chamber 4′ storing a first reactant and an indication chamber 4″ storing a second reactant. On the inner side of the closing foil 5 facing the infusion head 1, a separator 6 can be attached to the closing foil. The separator 6 can be sealed to the closing foil in the same manner as the foil can be sealed to the storing unit 2. The separator 6 can act on the monitoring device at a connecting conduit 7 connecting the activating chamber 4′ and the indication chamber 4″ and can block the connecting conduit 7 to separate the chambers 4′ and 4″.

In FIG. 1b, the medical assembly is shown from another side of FIG. 1a, which can be turned about 90°. As shown the separator 6 can be a fork which can pick up the monitoring device 4 between the activating chamber 4′ and the indication chamber 4″. The separator 6 can separate the reactants within these chambers such that they cannot react or interact with each other. In this state, the monitoring device 4 can be in an inactive state. The infusion head 1 can be safely stored within the storing unit 2 and may be stored for a long time in this state.

In FIG. 1c, the storing unit 2 can be opened by lifting the closing foil 5 for withdrawing the infusion head 1 from the storing unit 2 for when the infusion head 1 is to be applied to a medical device, for example, an infusion pump device. The storing unit 2, respectively the closing foil 5, can be moved relative to the disposable or replaceable infusion head 1 from the first position shown in FIGS. 1a-b to a second position shown in FIG. 1c. By moving the closing foil 5, which can mean lifting the foil 5 from the storing unit 2, the separator 6 can disengage from the monitoring device 4 of the infusion head 1 and can free the connecting conduit 7 between the activating chamber 4′ and the indication chamber 4″. The reactant of the activating chamber 4′ may flow into the indication chamber 4″ and can successively react with the reactant in the indication chamber 4″. This can mean the monitoring device 4 can be in an active state. The reaction may cause a change of color, which can slowly move in one direction within the indication chamber 4″. The moving color change may indicate the period of time elapsed since the opening of the storing unit 2 and can indicate the time remaining until the infusion head 1 has to be disposed. As soon as the storing unit 2 is opened to retract the infusion head 1 for application with an infusion device, the monitoring device 4 can automatically be started. No additional activation step may be necessary for activating the monitoring device 4.

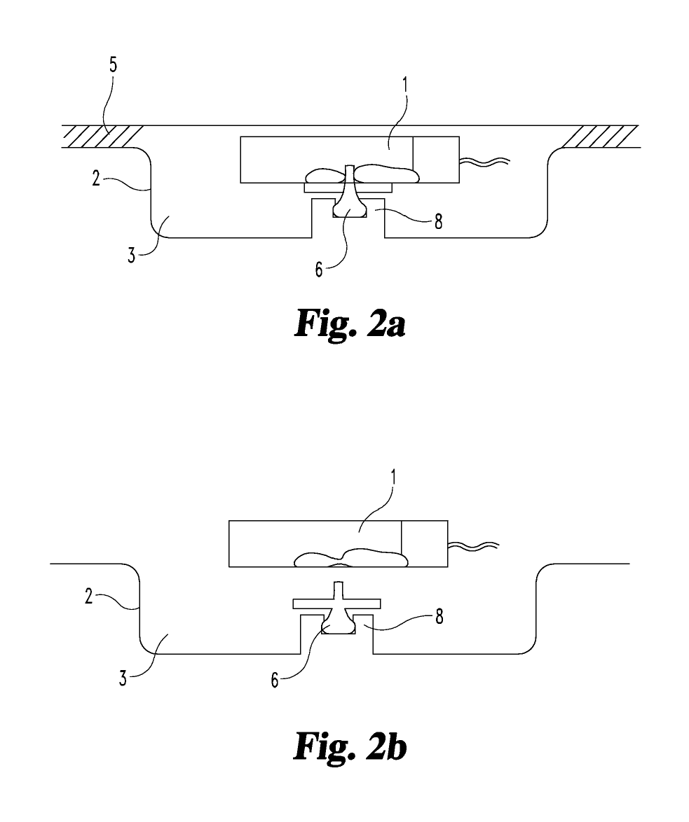

In FIGS. 2a and 2b, a second embodiment of a medical assembly is disclosed comprising a disposable or replaceable infusion head 1 and a storing unit 2 moveable relative to each other. The storing unit 2 can be closed with a closing foil 5. On the base of the storage compartment 3, a separator 6 can be attached by a snap engagement with a socket 8 on the base for holding tightly the separator 6. The separator 6 again can prevent the reactant of the activating chamber 4′ to flow into the indication chamber 4″ in this inactive state of the monitoring device 4 as explained in FIGS. 1a-c.

In FIG. 2b, the closing foil 5 has been removed from the storing unit 2 and the infusion head 1 can be withdrawn from the storage compartment 3 to apply the infusion head 1 to an infusion device. By taking out the infusion head 1 of the storage compartment 3, the infusion head 1 can be moved relative to the storing unit 2 and the monitoring device 4 can be detached from the separator 6, while the separator 6 can keep engaged with the socket 8. Thus the reactants of the activating chamber 4′ and indication chamber 4″ can be free to react with each other and the monitoring device 4 can be in an activated state. This can mean as soon as the infusion head 1 is taken out of the storing unit 2 to prepare the infusion device for application of infusion liquid, the monitoring device 4 can be activated automatically at the same time.

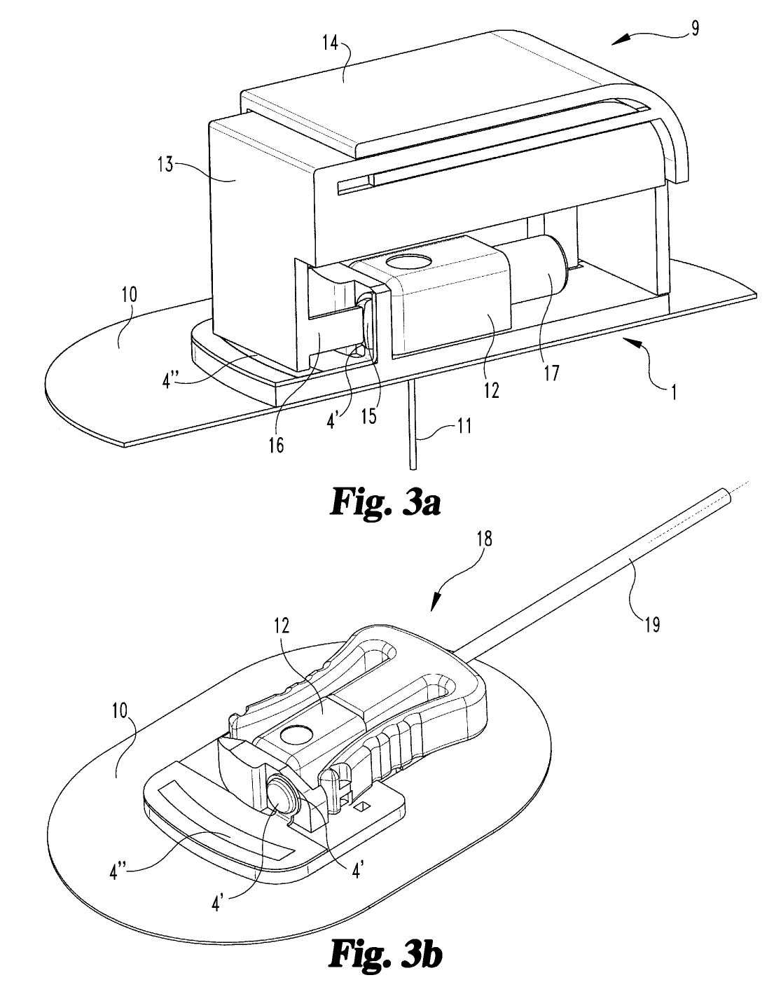

In FIG. 3a, still another medical assembly is shown comprising a disposable or replaceable infusion head 1 and an operation housing 9 moveable relative to the infusion head 1. The infusion head 1 can comprise a base pad 10, which can be attached to a body tissue of a user. Further the infusion head 1 can be provided with a cannula 11 moveably arranged on a cannula casing 12. The casing 12 can comprise a coupling port 17 in fluidic connection with the cannula 11. The operation housing 9 can sit on the base pad 10 and can enclose the cannula casing 12 and the coupling port 17. The casing 12 can house a cannula mechanism designed for moving the cannula 11 relative to the infusion head 1 from a storing position to an insertion position. Therefore, the operation housing 9 can comprise a sliding portion 13 and a fixed portion 14. The fixed portion 14 can be fixed relative to the base pad 10, while the sliding portion 13 can be slideably arranged relative to the fixed portion 14 and to the infusion head 1 and may interact with the cannula mechanism. As soon as the sliding portion 13 is pushed into the fixed portion 14, the sliding portion 13 can act on a pusher actuating the cannula mechanism, which in turn can move the cannula 11 from the storing position inside the casing 12 to an extended insertion position ready for insertion into a tissue a shown in FIG. 3a.

The pusher of the cannula mechanism can comprise an interaction area 15, which can be pressed by a ram 16 located on the sliding portion 13. The interaction area 15 can be covered by an activating chamber 4′ of the monitoring device. An indication chamber 4″ can be on the base pad 10 and can be separated from the activating chamber 4′ in a first state, in which the pusher of the cannula mechanism may not be under pressure of the ram 16 of the sliding portion 13. As soon as the sliding portion 13 presses against the interaction area 15 and the activating chamber 4′ as shown in FIG. 3a, the generated pressure in the activating chamber 4′ can result in unblocking a conduit to the indication chamber and pressing the reactant in direction of the indication chamber 4″ through a connection layer. Thus the two reactants may react in the indication chamber 4″ and the monitoring device can be in an activated state.

In a first position of the sliding portion 13, in which the sliding portion 13 can extend out of the fixed portion 14, the sliding portion 13 can cover the indication chamber 4″ on the base pad 10. The indication chamber 4″ may also be covered in a second position, in which the sliding 13 portion can be pushed into the fixed portion 14 and the pusher can be pressed. The operation housing 9 can be detached from the base pad 10 and be disposed. Therefore, the operation housing 9 can also be regarded as a disposable unit.

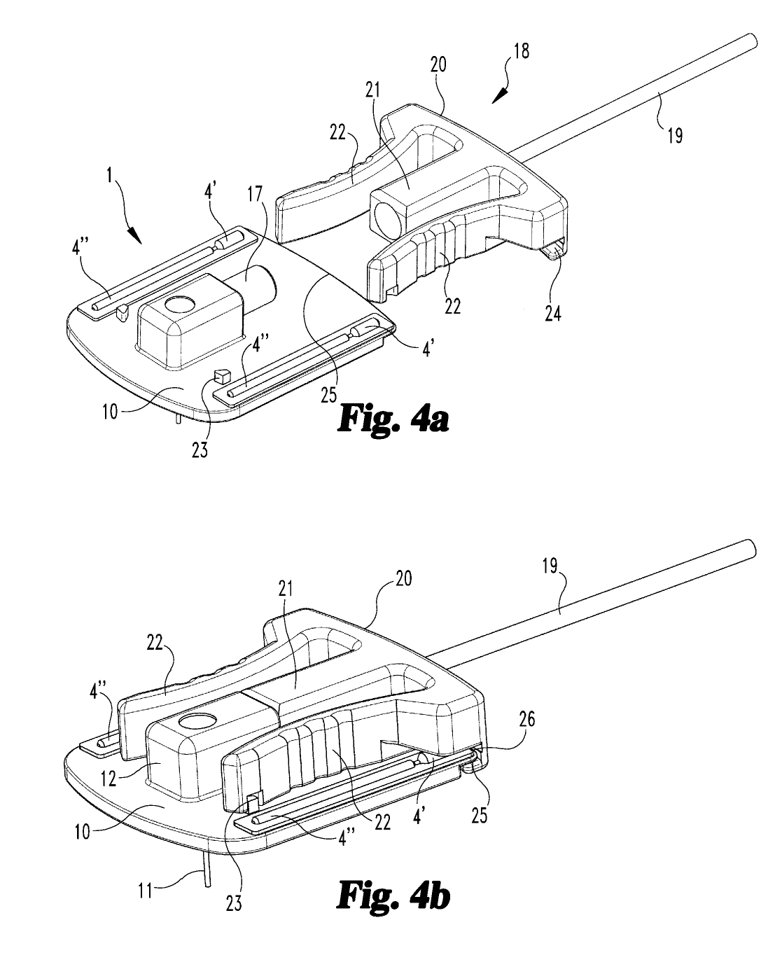

As soon as the operation housing 9 is removed of the infusion head 1, the casing 12 and the coupling port 17 can be accessible and also the indication chamber 4″ can be fully visible as seen in FIG. 3b. The coupling port 17 can be connected to a coupler 18 comprising an infusion tube 19. The coupling may for example be an attachment mechanism, which will be explained in more detail in FIGS. 4a-b. Thus the infusion head 1 can be connected to a medical device, the infusion device in this embodiment. The infusion tube 19 can be in fluidic connection with the cannula via the coupling port 17 and casing 12. The indication chamber 4″ can extend in form of a longitudinal strip on one side of the base pad 10 nearly over the full length of the base pad. The reactant of the activating chamber 4′ can travel to the reactant of indication chamber 4″ resulting in reaction of the two reactants so that a color indication can be realized, which can travel along the length of the indication chamber 4″. The indication chamber 4″ can indicate the time period remaining until disposal of the infusion head 1 by a color indication.

In FIGS. 4a-b, another embodiment of the medical assembly is illustrated comprising a disposable or replaceable infusion head 1 and a coupler unit in form of the coupler 18 moveable relative to the infusion head 1 to connect an infusion device to the infusion head 1 and can prepare the infusion device for application. The coupler 18 can comprise a main body 20 with a connection port 21 and a resilient attachment arm 22 on each side of the port 21 extending substantially parallel along the axis of the infusion tube 19 towards the infusion head 1. The attachment arms 22 can snap between attachment pins 23 extending upward from the base pad 10. The attachment arms 22 can snap into a fixation position with the pins 23 and can engage behind the pins 23 as can be seen in FIG. 4b. The main body 20 can comprise a groove 24 running substantially parallel to a positioning edge 25 of the base pad 10. A monitoring device 4 can comprise two activating chamber 4′ and two indication chamber 4″. The two activating chambers 4′ can be located close to the positioning edge 25 while the indication chambers 4″ can reach over the length of the base pad 10 on two opposing sides of the base pad 10 substantially perpendicular to the positioning edge 25.

In FIG. 4a, the infusion head 1 and the coupler 18 can take a first position relative to each other. The coupling port 17 and the connection port 21 can be separated. The monitoring device 4 can be in an inactive state. In FIG. 4b, the coupler 18 can be attached to the infusion head 1. Therefore, the coupler 18 can slide upon the base pad 10 of the infusion head 1 such that the attachment arms 22 can engage with the attachment pins 23 and the positioning edge 25 can slide into the groove 24 such that the surface of the main body 20 can press on the activating chambers 4′ on both sides of the pad 10. Therefore the main body 10 may comprise an inclined plane 26 along which the positioning edge 25 can be guided into the groove 24, and which can squeeze the activating chambers 4″ in the attachment position. This can mean the infusion head 1 and the coupler 18 can take a second position, the monitoring device 4 can be in an active state and the medical assembly can be connected to an infusion device and ready for use. The generated pressure in the activating chambers 4″ can cause reactant in the activating chambers 4″ to pass to the indication chambers 4″ and can react with the reactant in the indication chambers. Thus an indication line can move along the length of both indication chambers 4″ and can indicate a period of time for example for replacing the infusion head 1 on the coupler 18. The two indication chambers 4″ on both sides of the casing 12 and the coupler 18 can enable a user to view the monitoring device irrespective of the orientation of the infusion head 1 on a body tissue. The monitoring device can be automatically activated, as soon as the coupler 18 is attached to the infusion head 1 to prepare the infusion device for application.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A disposable medical device for use in combination with a further medical device, the medical device comprising:
a monitoring device for counting and/or displaying a time period of application and/or time remaining before required disposal or replacement of the disposable or replaceable unit, the monitoring device switches, upon activation, from an inactive state to an active state where its counts and/or displays the time period of application and/or time remaining before required disposal,
wherein the monitoring device is automatically activated along with carrying out a preparatory or handling step for preparing the disposable medical device for use.

2. The disposable medical device according claim 1, wherein the monitoring device is pressure sensitive.

3. The disposable medical device according to claim 1, wherein the monitoring device comprises an interaction area automatically activated along with carrying out the preparatory or handling step.

4. The disposable medical device according to claim 1, wherein the monitoring device comprises at least one activating chamber including a first reactant and at least one indication chamber including a second reactant, wherein the activating chamber and the indication chamber are separated before carrying out the preparatory or handling step and are connected after carrying out the preparatory or handling step.

5. The disposable medical device according to claim 4, wherein after carrying out the preparatory or handling step, first and second reactants react with each other such that a visible indication moves along an axis of the indication chamber in a predetermined period of time.

6. The disposable medical device according to claim 1, wherein the monitoring device is sealed onto a surface of the disposable medical device.

7. The disposable medical device according to claim 1, wherein the disposable medical device is as an infusion head to be placed on tissue of a patient for administration of medical or pharmaceutical liquid into the tissue comprising a connected or connectable cannula.

8. The disposable medical device according to claim 7, wherein an indication chamber is provided on at least two opposing sides of the infusion head.

9. The disposable medical device according to claim 7, further comprising,
a housing, which at least partly covers the infusion head, is moveable relative to the cannula and interacts with the cannula of the infusion head.

10. A medical assembly comprising at least two units moveable relative to each other, wherein at least one of the units is a disposable unit or device designed for application in combination with a medical device and a chemical or physical monitoring device for counting and/or displaying a time period of application and/or time remaining before required disposal or replacement of the disposable unit, wherein in an inactive state, the at least two units take a first position relative to each other and the monitoring device is inactive and in an active state, the at least two units take a second position relative to each other and the monitoring device is active, wherein the monitoring device is automatically activated to count and/or display the time period by movement of the at least two units relative to each other from the first to the second position.

11. The medical assembly according to claim 15, wherein the monitoring device is a disposable medical device according to claim 1.

12. The medical assembly according to claim 10, wherein the first or second unit acts on the interaction area and activates the monitoring device, when moved from first to second position.

13. The medical assembly according to one of claim 10, wherein the second unit is a storage unit for storing the first unit comprising a separator for separating the activating chamber and the indication chamber.

14. The medical assembly according to claim 13, wherein the separator is on a base of the storing unit, wherein the monitoring device of the disposable unit rests on the separator in the first state.

15. The medical assembly according to claim 13, wherein the separator is on a closing foil of the storing unit and engages with the monitoring device in a closed state of the storing unit.

16. The medical assembly according to claim 10, wherein the disposable unit is an infusion head and the second unit is a preparation or storing unit interacting with the infusion head.

17. The medical assembly according to claim 16, wherein the second unit is realized by a coupler releasably connectable to a coupling port of the infusion head.

18. A method of preparing at least part of a medical device for application, wherein the medical device comprises at least one disposable unit or device and a chemical or physical monitoring device for counting and/or displaying a time period of application and/or time remaining before required disposal or replacement of the disposable unit, the method comprising:
automatically activating the monitoring device by moving the at least one disposable unit relative to a second unit;
preparing or positioning the disposable unit for use with the medical device;
assembling the at least one disposable unit and the medical device; and
withdrawing the disposable unit of a storage compartment of the second unit.

19. The method according to claim 18, wherein the medical device is an infusion device comprising a disposable unit in form of an infusion head, and wherein the monitoring device is activated by,
connecting the infusion head to a coupler of the infusion device;
preparing or positioning a cannula moveably arranged on/at the infusion head; or
opening a storage unit housing the infusion head, or withdrawing the infusion head of the storage unit.
